SEQUENCE LISTING (14 sequences)

SEQ ID NO: 1 - protein, 1141 aa, Homo sapiens [sequence omitted]

SEQ ID NO: 2 - DNA, 4249 bp, Homo sapiens
ccgtcggtgaggcggtgccgggcgggggttgtcgggtgtcatgggcggtggcgacggcaccgcccccgcgtctccctgagcgggacggcagggggggcttctgcgctgagccgggcgatggacgacagcggcgagctgggtggtctggagaccatggagaccctcacggagctgggcgacgagctgaccctgggagacatcgacgagatgctgcaatttgtcagtaatcaagtgggagagttccctgacttgttttcagaacagctgtgtagctcctttcctggcagtggtggtagtggtagcagcagcggcagcagtggcagcagcagcagcagcagcaatggcaggggcagcagcagcggagctgtggacccttcagtgcaacggtcattcacccaggtcacattaccttccttctctccctcggcggcctccccacaggctccaactctgcaagtcaaggtttctcccacctcagttcccaccacacccagggcaactcctattcttcagccccgcccccagccccagcctcaacctcaaactcagctgcaacaacagacggtaatgatcacgccaacattcagcaccactccgcagacgaggatcatccagcagcctttgatataccagaatgcagctactagctttcaagtccttcagcctcaagtccaaagcctggtgacatcctcccaggtacagccggtcaccattcagcagcaggtgcagacagtacaggcccagcgggtgctgacacaaacggccaatggcacgctgcagacccttgccccggctacggtgcagacagttgctgcgccacaggtgcagcaggtcccggtcctggtccagcctcagatcatcaagacagattcccttgttttgaccacactgaagacagatggcagccctgttatggctgcggtccagaacccggccctcaccgccctcaccacccctatccagacggctgcccttcaagtaccaaccctggtgggcagcagtgggaccattctgaccacaatgcctgtaatgatggggcaagagaaagtgcccattaagcaggtacctgggggagtcaagcagcttgagccccccaaagaaggagaaaggcggacaacccataatatcattgagaaacgatatcgctcctccatcaatgacaaaatcatcgaattgaaagacctggtcatggggacagacgccaagatgcacaagtctggcgttctgaggaaggccattgattacatcaaatacttgcagcaggtcaatcataaactgcgccaggagaacatggtgctgaagctggcaaatcaaaagaacaagcttctaaagggcatcgacctaggcagtctggtggacaatgaggtggacctgaagatcgaggactttaatcagaatgtccttctgatgtcccccccagcctctgactcagggtcccaggctggcttctctccctactccattgactctgagccaggaagccctctattggatgatgcaaaggtcaaagatgagccagactctcctcctgtggcgctgggcatggtagaccgctcacggattcttctgtgtgtcctcaccttcctgtgcctctcctttaaccccctgacttccctgctgcagtggggaggggcccacgactctgaccagcacccacactcaggctctggccgcagtgtcctgtcattcgagtcaggttctgggggctggtttgactggatgatgcctactcttctcttatggctggtaaatggtgtgattgtcctgagcgtctttgtgaagctgctggttcatggggagccagtgatccggccacactcgcgctcctcggtcaccttctggaggcaccggaaacaggcagatctggatctcgccagaggagattttgcagctgctgccgccaacctacaaacctgcctggcagttttgggccgggcactgcccacctcccgcctggacctggcctgcagcctctcctggaacgtgatccgctacagcctgcagaagctacgcctggtgcgctggctgctcaagaaagtcttccagtgccggcgggccacgccagccactgaggcaggctttgaagacgaagctaagaccagcgcccgggatgcggctctggcctatcaccggctgcaccagctgcacatcacagggaagcttcctgcaggatccgcctgttccgatgtacacatggcgttgtgtgccgtgaacctggctgaatgtgcagaggagaagatcccaccgagcacactggttgagatccatctgactgctgccatggggctcaagacccggtgtggaggcaagctgggcttcctggccagctacttcctcagccgagcccagagcctgtgtggccccgagcacagtgctgttcctgactccctgcgctggctctgccaccccctgggccagaagtttttcatggagcggagctggtctgtgaagtcagctgccaaggagagtctatactgtgcccagaggaacccagctgaccccattgcgcaggtccaccaggccttctgcaagaacctgctggagcgagctatagagtccttggtgaaacctcaggccaagaagaaggctggagaccaggaagaagagagctgtgaattctccagtgctctggagtacttgaaattacttcattcttttgtggactctgtgggggttatgagccccccactctccaggagctccgtgctcaagtccgccctgggtccagacatcatctgtcggtggtggacgtctgcaatcactgtggccatcagctggctccagggagacgatgcagctgtgcgctctcattttaccaaagtggaacgcatccccaaggccctggaagtgacagagagccccctggtgaaggccatcttccatgcctgcagagccatgcatgcctcactccctgggaaagcagatgggcagcagagttccttctgccattgcgagagggccagtggccacctatggagcagcctcaacgtcagtgggggcacctctgaccctgccctcaaccacgtggtccagctgctcacctgtgacctgctactgtcgctacggacagcgctctggcaaaaacaggccagtgccagccaggctgtgggggagacctaccacgcgtcaggcgctgaactggcgggcttccaacgggacctgggcagcctgcgcaggctggcacacagcttccgcccagcataccgcaaggtgttcctgcatgaagccaccgtgcgcctgatggcaggaggcagccccacccgcacccaccagctgctggaacacagcctgcggcggcgcaccacgcagagcaccaagcacggagaggtggatgcctggcccggccagcgagagcgggccaccgccatcctgctggcctgccgccacctgcccctctccttcctctcctccccgggccagcgggcagtgctgctggccgaagctgcccgcaccctggagaaggtgggcgaccggcgctcctgcaacgactgccagcagatgattgttaagctgggtggtggcactgccattgccgcctcctgaccaccaggctcagcccacccctccacctctctctcgatttctctctctccccctcagcatcttcccgctgagagtggtggggaagagccttgtcttcttagctgtcacctgccgaggcttctgggccactcaggccagtgcacccctgggcagagccccttaaagctgctgtcactagatgcccatggtccagggcctggtgggcgtgagaggataggtggcagggcagaaactgggcagccctgacttgatagcagcagggggagctcccaagctgccaagcccctgcctccagccttcctgagtttctctctcctgaaccctactctctcctttttgcttcctcagtttttatcaggctttctctgggggacagcagtctctgagcaccagggagcagttgccctcaggcctgtgcccagcatgccctcccctttttatacgaatgttttctaccagtgtgcttgggtttgccatgatgcgaggctgagttgctgtagcgtcttgattctctccctgggtctgcgttccctcccctgggcctgactgagcctgctcattgtttttccctttattacacaggacagccaggggaggaggggggcccagccctgggaggctggtgggaggcagggggcaggcctgcggatgcatgaaataatgttggcattattttttaattttttaaaaaataaatggtatctta

SEQ ID NO: 3 - DNA, 26 bp, Artificial
  Synthetic primer
tctcaactccttgaaaatagctacct

SEQ ID NO: 4 - DNA, 26 bp, Artificial
  Synthetic primer
ggtctatgattgtagctggtcactta

SEQ ID NO: 5 - DNA, 23 bp, Artificial
  Synthetic primer
agcttctaaagggcatcgaccta

SEQ ID NO: 6 - DNA, 28 bp, Artificial
  Synthetic primer
tagaggacacctagtcagacaaaatgat

SEQ ID NO: 7 - DNA, 24 bp, Artificial
  Synthetic primer
ccaactctgcaagtcaaggtttct

SEQ ID NO: 8 - DNA, 23 bp, Artificial
  Synthetic primer
gcgtgatcattaccgtctgttgt

SEQ ID NO: 9 - DNA, 20 bp, Artificial
  Synthetic primer
aggaaacttcgctcacacct

SEQ ID NO: 10 - DNA, 20 bp, Artificial
  Synthetic primer
gccatgtatctgttttggcc

SEQ ID NO: 11 - DNA, 29 bp, Artificial
  Synthetic primer
cagcagcccagcagaggttttctacaatc

SEQ ID NO: 12 - DNA, 23 bp, Artificial
  Synthetic primer
tggctcacagttcttcataacca

SEQ ID NO: 13 - DNA, 23 bp, Artificial
  Synthetic primer
atgacatccttcagtggcttgtc

SEQ ID NO: 14 - DNA, 26 bp, Artificial
  Synthetic primer
tccatgccctctagaactttgccgaa

(19) United States
(12) Patent Application Publication
(10) Pub. No.: US 2009/0320147 A1
(43) Pub. Date: Dec. 24, 2009

(54) Nonhuman transgenic animal as type 2 diabetes model

(76) Inventors: Hitoshi Shimano, Ibaraki (JP); Nobuhiro Yamada, Ibaraki (JP)

Correspondence Address: MORGAN LEWIS & BOCKIUS LLP, 1111 PENNSYLVANIA AVENUE NW, WASHINGTON, DC, 20004, US

(21) Appl. No.: 11/547,663

(22) PCT Filed: Apr. 8, 2005
(86) PCT No.: PCT/JP05/07285

§ 371 Date: Sep. 29, 2008

(30) Foreign Application Priority Data
Apr. 9, 2004 (JP) 2004-115646

(51) Int. Cl. G01N 33/50; A01K 67/027; C12N 15/87

(52) U.S. Cl. 800/3; 800/9; 800/25

(57) ABSTRACT

The invention provides a non-human transgenic animal as a model of type 2 diabetes manifesting a symptom of type 2 diabetes by excessive expression of the active SREBP-2 protein in pancreatic β-cells by introducing a recombinant DNA in which a DNA encoding the active SREBP-2 protein is disposed under the control of a promoter, and a method for screening therapeutic agents of diabetes using the transgenic animal.

### TECHNICAL FIELD

This invention relates to a non-human transgenic animal as a type 2 diabetes model, a method for screening therapeutic agents for diabetes using the model animal, and a method for preparing the type 2 diabetes model animal.

### BACKGROUND ART

Diabetes is called as a contemporary national disease as a central core of lifestyle-related diseases, and developments of preventive and therapeutic methods thereof are problems to be urgently solved. While the mechanism of onset of the disease has not been elucidated yet, it is considered that two disease conditions of deficiency of insulin as a hormone for lowering the blood glucose level (insufficiency of insulin secretion) and impairment of the insulin action (insulin resistance) are intermingled. Usually, diabetes is classified into: (1) type 1 requiring continuous replenishment of insulin since β-cells of the pancreas for producing insulin are destroyed; (2) type 2 in which secretion of insulin is deficient or action of insulin is deteriorated; (3) other diabetes by specific causes; and (4) gestational diabetes and the like.

Type 1 diabetes is one of autoimmune diseases, and is clinically called as insulin dependent diabetes. The β-cells of the pancreas that secrete insulin are destroyed by being attacked by the autoimmune system. Since insulin is a hormone for allowing blood glucose to be absorbed by cells to decrease the blood glucose level, glucose in the blood increases while glucose in the cell becomes deficient when the amount of secretion of insulin decreases. The cells cannot maintain their life activities when such glucose deficient state persists, and various impairments of organs, loss of sight and necrosis of foots are caused. The model mouse of type 1 diabetes is known in the art, and studies on the therapy of type 1 diabetes have been advanced using a mouse model (for example, see Science, 2003, Nov. 14; 302 (5648): p 1223-7).

Type 2 diabetes is often called as insulin independent diabetes from the clinical point of view, and the disease is developed by insufficient insulin secretion from the pancreatic β-cells and by insulin resistance. Whether insufficient insulin secretion or insulin resistance is strongly concerned with type 2 diabetes is different depending on respective cases or the process of the case, both types of causes are often intermingled. While insulin is instantaneously secreted in response to the start of increase of the blood glucose level by absorbing glucose after the meal in normal person, this response is lacking in the insulin secretion deficient case, and insulin is secreted by being retarded from the increase of blood glucose level. Insulin resistance refers to a state in which the action of insulin is impaired by various reasons, although insulin is secreted. The immunological mechanism of type 1 diabetes is clear, and a model mouse expressing the disease condition has been already reported as described above. However, since the cause of type 2 diabetes is complicated, mouse model capable of sufficiently describing the human disease condition has not been reported yet.

On the other hand, SREBP (Sterol Regulatory Element Binding Protein) family belongs to s transcription factor family for controlling sterol synthesis, and binds to a specific sequence SRE common to a cholesterol synthesis enzyme group and LDL receptor gene promoter region for controlling expression of transcription. SREBP is known to include two subfamilies of SREBP-1 (SREBP-1a and SREBP-1c) and SREBP-2. Former studies have revealed that SREBP-1c is responsible for the synthesis of fatty acids and neutral lipids (triglycerides), and SREBP-2 is responsible for the synthesis of cholesterol (J. Clin. Invest. 1996, 98, 1575-1584; J. Clin. Invest. 1997, 99: 846-854; J. Clin. Invest. 1998, 101:2331-2339; Cell, Review, 1997; 331-40). However, there have been no reports on the relation between SREBP-2 and diabetes, particularly between SREBP-2 and type 2 diabetes.

Japanese Patent Application National Publication No. 2003-501102 discloses an animal model of a genetically modified fly or threadworm for expression or abnormal expression of SREBP protein. However, the experimental animal described in this patent publication (fly or threadworm) is developed for use in the study on lipid metabolism. This patent publication does not suggest the relation between SREBP protein and diabetes at all.

### DISCLOSURE OF INVENTION

The number of diabetes patients in this country is estimated to be from 6 to 7 million, and the number of latent patients is supposed to be approximately the same. However, no mechanism of onset of the disease has been elucidated, and no effective therapeutic agents have been developed today. In particular, since the proportion of type 2 diabetes is as large as from 90 to 97%, developments of therapeutic methods and therapeutic agents have been strongly desired.

The inventors of the invention have developed a mouse capable of specifically and forcibly expressing SREBP-2 in the pancreatic β-cells that are insulin-secreting cells by introducing a DNA encoding the human active SREBP-2 protein in mouse. Although an expected change has been variation of metabolism of cholesterol (activation of synthesis) in the pancreatic β-cells, surprisingly insulin secretion is remarkably reduced with an increase of the blood glucose level in addition to abnormal metabolism of cholesterol, and a symptom resembling to type 2 diabetes is found to be manifested. Such disease condition is different from the action mechanism according to conventional theories of lipid toxicology that secretion of insulin decreases by a fatty acid excess state and accumulation of neutral lipids (triglycerides) in the cell. In other words, variation of cholesterol metabolism or SREBP-2 itself may be a cause of type 2 diabetes. This discovery is quite epoch-making considering that no reports have been found on the relation between the decrease of insulin secretion and diabetes related to the metabolism of cholesterol.

The invention completed based on the above-mentioned discoveries provides a non-human transgenic animal as a model of type 2 diabetes, a method for screening a therapeutic agent of diabetes using the model animal, and a method for preparing the model animal for type 2 diabetes as follows:

(1) a non-human transgenic animal as a model of type 2 diabetes manifesting a symptom of type 2 diabetes by excessive expression of the active SREBP-2 protein in pancreatic β-cells by introducing a recombinant DNA in which a DNA encoding the active SREBP-2 protein is disposed under the control of a promoter;

(2) the transgenic animal according to (1), wherein the symptom of type 2 diabetes includes abnormal cholesterol metabolism and impaired insulin secretion in the pancreatic β-cells;

(3) the transgenic animal according to (1), wherein the DNA encoding the active SREBP-2 protein is a human active SREBP-2 cDNA;

(4) the transgenic animal according to (1) or (2), wherein the promoter is a promoter of rat insulin I gene;

(5) the transgenic animal according to any one of (1) to (4), wherein a gene marker is further introduced into the recombinant DNA;

(6) the transgenic animal according to (5), wherein the gene marker is a green fluorescent protein;

(7) the transgenic animal according to any one of (1) to (6), wherein the animal is mouse, rat or rabbit;

(8) the transgenic animal according to (7), wherein the animal is mouse;

(9) a method for screening a therapeutic agent for type 2 diabetes using the transgenic animal according to any one of (1) to (8);

(10) the method for screening a therapeutic agent for type 2 diabetes according to (9) comprising the step of observing the change of the symptom of type 2 diabetes after administering a test compound to the transgenic animal; and

(11) a method for preparing a transgenic animal manifesting the symptom of type 2 diabetes comprising the steps of: constructing a recombinant DNA in which a DNA encoding a human active SREBP-2 protein is disposed under the control of a promoter; introducing the recombinant DNA and a gene marker into fertilized ovum of a non-human animal; transplanting the fertilized ovum into a pseudo-pregnant non-human mammal to breed the mammal; and selecting infants having the recombinant DNA from the delivered infants using an expression product of the gene marker as an index.

The transgenic animal of the invention as a novel model animal of type 2 diabetes is useful as a tool for studying the mechanism of onset of diabetes and for screening a therapeutic agent of diabetes. While many other existing model animals of diabetes are required to have high lipid diet as an essential condition of onset of diabetes or the animals are evidently obese, the transgenic mouse according to the preferred aspect of the invention spontaneously develops diabetes without being obese, and the disease condition resembles to the disease condition of type 2 diabetes dominant in Japanese. Accordingly, the transgenic mouse according to the invention is useful as a model mouse of type 2 diabetes manifesting above-mentioned disease conditions.

### BRIEF DESCRIPTION OF THE DRAWINGS

FIG. 1 shows a construction of the SREBP-2 gene expression vector.

FIG. 2 is a graph showing the fasting blood glucose level and fasting insulin level in the SREBP-2 transgenic mouse.

FIG. 3 is a graph showing the changes of the blood glucose level and of the blood insulin level in an intravenous glucose loading test.

FIG. 4 shows insulin secretion ability by glucose stimulation of isolated Langerhans' islet.

FIG. 5 shows a comparison of the amounts of expression between transgenic human SREBP-2 and HMGCoA synthetase.

### BEST MODE FOR CARRYING OUT THE INVENTION

### 1. Non-Human Transgenic Animal as a Model Animal of Type 2 Diabetes

The invention provides a novel model animal type 2 diabetes that decreases insulin secretion ability by allowing pancreatic β-cells to excessively express a cholesterol synthesis control transcription factor SREBP-2. Such a model animal is useful for elucidating the mechanism of onset of type 2 diabetes and for developing therapeutic agents.

Specifically, a first aspect of the invention provides a non-human transgenic animal as a model of type 2 diabetes, wherein an introduced recombinant DNA having a DNA encoding an active SREBP-2 protein is disposed under the control of a promoter, and the animal manifests symptoms of type 2 diabetes by forced excessive expression of SREBP-2 in the pancreatic β-cells.

The “SREBP-2 protein” as used in the present specification is one of SREBP subfamily as a sterol regulatory element binding protein, and a known membrane-bound protein related to regulation of cholesterol synthesis. The SREBP-2 gene and SREBP-2 protein are described, for example, in Proc. Natl. Acad. Sci. USA, 1993, Dec. 15; 90(24): 11603-7. In particular, the amino acid sequence of the human SREBP-2 protein is deposited with NCBI and given Accession No. AAA50746, and is represented by SEQ ID No. 1. The base sequence of the human SREBP-2 cDNA is deposited with GenBank and is given Accession No. U02031, and is expressed by SEQ ID No. 2.

Endogenous expression of SREBP-2 is widely distributed in almost all tissues, although the extent of expression is a little different in respective tissues. However, SREBP-2 is present as a membrane-bound protein that is quite unique as a transcription factor, and cannot directly exhibit its activity as the transcription factor. The active part is cleaved only when cholesterol requirement of the cell is enhanced, transferred into nuclei, and the cell supplies cholesterol by enhancing expression of a group of genes of a cholesterol synthesis system. On the contrary, the transcriptional activity of SREBP-2 is not exhibited without cleaving the active part when cholesterol is abundant. This system permits the amount of cholesterol in the cell to be appropriately controlled.

Since the control mechanism of cholesterol caused by the SREBP-2 is a system usually working in all the cells, the same mechanism is supposed to be valid in normal β-cells. Since only the portion transferred into the nucleus, or active SREBP-2, is forcibly and excessively expressed in the transgenic animal of the invention, above-mentioned feedback is not valid and cholesterol synthesis is forcibly in an enhanced state. In other words, the activity is maintained to be specific to the β-cell in the transgenic animal of the invention to consequently manifest the symptom of type 2 diabetes. Examples of symptoms of type 2 diabetes include abnormal metabolism (excess synthesis) of cholesterol and impairment of insulin secretion in the pancreatic β-cell as well as hyperglycemia, positive urine sugar and no occurrence of obesity. These symptoms can be judged by a skilled person in the art by known methods. For example, hyperglycemia and impaired insulin secretion can be judged by investigating the tendency of hyperglycemia and decline of insulin secretion in the glucose load test. Whether these symptoms are manifested or not may be judged by those skilled in the art. For example, excess synthesis of cholesterol may be judged by an increase of the amount of cholesterol of 10% or more, more definitely 20% or more, and further definitely 30% or more in the model animal group as compared with a normal animal group, when the Langerhans' islet is isolated from the pancreas of mouse and lipids are extracted to measure the content of cholesterol. Impaired insulin secretion can be judged by a decrease of the amount insulin of 20% or more, more definitely 30% or more, in the culture medium of the model animal group as compared with the normal animal group, when the Langerhans' islet is isolated from the pancreas of mouse and the amount of insulin secretion is measured in a high glucose medium. Alternately, impaired insulin secretion due to decline of the insulin secretion ability in a living animal may be judged for each animal by a statistically significant decrease of the insulin concentration of the model animal group as compared with the normal animal group, when a mouse is subjected to the sugar load test, or the mouse is loaded with glucose, the blood of the mouse is sampled with time, and the blood insulin concentration is measured. Hyperglycemia may be judged by a statistically significant increase of the blood glucose level of the model animal group as compared with the normal animal group in the glucose load test or repeated sampling of the blood. Since diagnostic criteria for human cannot be simply applied for the diagnosis of diabetes of the animal, the animal may be diagnosed as diabetes when the blood glucose level is constantly high and urine sugar is positive. The decrease of insulin secretion ability also strongly supports the diagnosis of diabetes when the animal develops diabetes of declined insulin secretion type.

The “active SREBP-2 protein” as used in the present specification refers to an active part of the cleaved SREBP-2 protein as mentioned above or mutated proteins thereof. In more detail, the “active SREBP-2 protein” is a protein having amino acid residues of 1st to 460th amino acid sequences represented by SEQ ID No. 1 (human active type) or mutated proteins thereof, and has an activity as a transcription factor against cholesterol synthesis-related genes. While the active SREBP-2 protein of the invention may be derived from any species, it is preferably a protein derived from human. While the “mutated protein” is not particularly restricted so long as it has a function as a transcription factor against the cholesterol synthesis-related genes, the protein is a mutated protein comprising, for example, deletion, substitution, insertion and/or addition to the amino acid sequence of the human protein. While the mutation site and number of the amino acids are not particularly restricted so long as the mutated protein maintains the transcription activity, the number of mutation is usually at 1 to 30 amino acid residues, preferably at 1 to 10 amino acid residues, and more preferably at 1 to several amino acid residues (for example 6 amino acid residues).

While the animal species as the object of the invention is not particularly restricted, examples of the preferable animal include mouse, rat, rabbit, goat and cattle.

Mouse is most preferable among them since preparation of the mouse line is easy.

### 2. Preparation of Transgenic Animal

The invention also provides a method for preparing the transgenic animal.

Specifically, the method for preparing the transgenic animal of the invention comprises the steps of: (1) constructing a recombinant DNA in which a DNA encoding a human active SREBP-2 protein is disposed under the control of a promoter; (2) introducing the recombinant DNA and a gene marker into a fertilized ovum of a non-human animal; (3) transplanting the fertilized ovum obtained into a pseudo-pregnant non-human animal; (4) breeding the animal; and (5) selecting infants having the recombinant DNA in the chromosome from delivered infants using an expression product of the gene marker as an index, to select a line in which the protein derived from the recombinant DNA is stably expressed in a desired tissue after establishing the line by breeding.

The method for preparing the transgenic animal (for example mouse) is well known, and is described in detail in (1) Method in Enzymology, Vol. 225, “Guide to Techniques in Mouse Development”, edited by Paul M. Wassarman, Melvin L. DePamphilis, Academic Press, Inc.; (2) “Up-to-Date Technology of Gene Targetting”, Yodo-Sha Co.; and (3) Gordon, J. W., 1993, “Guide to Techniques in Mouse Development”, (Wassarman, P. M. and DePamphilis, M. L., Eds.), Academic Press, San Diego. PCR, production of primers, production of genome DNAs, cloning and enzyme processing methods can be performed by conventional methods well known to those skilled in the art (see “Molecular Cloning, A Laboratory Manual”, Third Edition, Cold Spring Harbor Press, Cold Spring Harbor Laboratory, Cold Spring Harbor, New York, 2001).

Since the animal preferably used in the invention is the mouse as described above, the method for preparing the non-human transgenic animal of the invention is described with reference to the method for preparing the transgenic mouse.

For preparing the transgenic mouse of the invention, the recombinant DNA (or a DNA construct) is constructed so that the active SREBP-2 gene used in the invention is disposed under the control of a promoter. The DNA encoding the active SREBP-2 protein used in the invention may be produced by many methods including DNA synthesis, cloning of cDNA, cloning of genomes, polymerase chain reaction (PCR) and a combination of these technologies (for example, see “Molecular Cloning, A Laboratory Manual”, Third Edition, Cold Spring Harbor Press, Cold Spring Harbor Laboratory, Cold Spring Harbor, N.Y., 2001).

The DNA encoding the active SREBP-2 protein used in the invention can be constructed by conventional gene recombination techniques. For example, the DNA is obtained by screening a cDNA library by a PCR method or hybridization method using a primer or a probe designed and synthesized based on information on known sequences of amino acids and bases. The SREBP-2 protein can be expressed in an appropriate host by forming a DNA construct or an expression vector produced by linking the desired DNA thus obtained at the downstream of an appropriate promoter. The DNA encoding the active SREBP-2 protein used in the invention is preferably a human active SREBP-2 cDNA.

Examples of the vector used in the invention include plasmids, cosmids and viruses (including phages). These vectors can be also designed and constructed using conventional gene recombination technologies. These vectors are usually linked in the direction from a 5′-end to a 3′-end so that the promoter and human active SREBP-2 gene can be expressed. While the promoter is not particularly restricted so long as the SREBP-2 gene is able to be specifically expressed to be specific to the pancreatic β-cell, examples of the promoter include the promoter of rat insulin I gene promoter (Alpert, S., Hanahan D, Teitelman G Cell, 1988, Apr., 22; 53(2): 295-308) and amylin promoter. The promoter of rat insulin I gene is particularly preferable in the invention. An enhancer, a silencer and a poly A addition signal may be integrated into the expression vector so that the desired gene is properly expressed.

It is preferable that the gene marker is introduced into the vector together with the DNA used in the invention. Examples of such gene marker include green fluorescent proteins (such as GFP and EGFP), luciferase, β-galactosidase and chloramphenicol acetyltransferase. When the gene marker is introduced into the expression vector together with the DNA, introduction of the recombinant DNA and gene marker and expression thereof may be efficiently confirmed by using an expression product of the gene marker expressed in an infant mouse as an index.

While examples of the method for introducing the recombinant DNA thus obtained into the fertilized ovum include a microinjection method, a retrovirus vector method or a method using embryonic stem cells, the microinjection method is usually used. The fertilized ovum cultivation method, microinjection method and transplantation method of the fertilized ovum are well known by those skilled to the art.

Subsequently, the fertilized ovum to which the recombinant DNA has been introduced by microinjection is transplanted to the uterine tube of the pseudo-pregnant mouse. The mouse is bred, and the transgenic mouse of the invention can be obtained by selecting an infant mouse having the recombinant DNA from the delivered mice.

Selection of the infant mouse having the recombinant DNA from the delivered infant mice can be confirmed by discriminating SREBP-2 produced by introducing the recombinant DNA from the endogenous SREBP-2 by PCR using a specific primer having the base sequence of the human SREBP-2 gene and a part of other base sequences necessary for expression using genome DNA extracted from the tail of the mouse. Otherwise, introduction of the recombinant DNA and gene marker may be confirmed using an expression product of the gene marker as an index, when the gene marker is introduced into an expression vector.

For judging whether the transgenic mouse of the invention can be used as a model mouse of type 2 diabetes or not, expression of the introduced gene in the pancreatic β-cell in the mouse is confirmed at first, and abnormal metabolism of cholesterol in the β-cell as a direct effect of gene introduction is investigated. Finally, impaired insulin secretion in pancreatic β-cell as well as phenotype of diabetes such as hyperglycemia, polyuria and positive urine sugar is confirmed.

### 3. Screening Method

The transgenic mouse prepared by the inventors of the invention can be concluded to be the model mouse of type 2 diabetes from the view point of clinical finding, clinical test and pathology as confirmed in the examples hereinafter. Accordingly, the transgenic mouse of the invention as the animal model of type 2 diabetes is useful as a screening tool for elucidating onset mechanisms of type 2 diabetes and for developing therapeutic methods and therapeutic agents thereof.

Another aspect of this invention provides a screening method of the therapeutic agent of type 2 diabetes comprising observing the change of symptoms of diabetes after administering a test compound to the transgenic animal. Specifically, the method for confirming the test compound to be effective or not as a therapeutic agent of type 2 diabetes comprises: grouping the transgenic mouse of the invention into test mouse groups and non-transgenic littermates into reference mouse groups; administering the test compound to the test mouse group; sampling the urine and blood from the test mouse group and reference mouse group to measure the content of glucose in the urine and blood glucose level; and comparing investigation these measured values.

### EXAMPLES

While the invention is described hereinafter with reference to examples and experimental examples, the invention is by no means restricted to these examples.

### (1) Production of SREBP-2 Gene Expression Vector Under the Control of Insulin Promoter

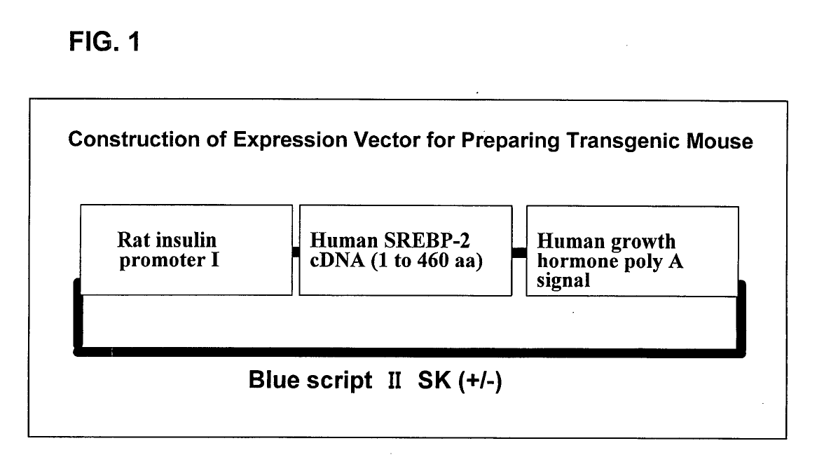

Standard methods were used for molecular biology methods (see “Molecular Cloning, A Laboratory Manual”, Third Edition, Cold Spring Harbor Press, Cold Spring Harbor Laboratory, Cold Spring Harbor, N.Y., 2001). For producing DNAs for microinjection necessary for preparing the transgenic mouse, SREBP-2 gene expression vectors were produced under the control of an insulin promoter. The construction thereof is shown in FIG. 1.

### (Promoter)

Rat insulin promoter I (Alpert, S., Hanahan, D. and Teitelman, G, Cell, 1988, Apr., 22; 53(2): 295-308) that has been successful for expression in the past Tg mouse was used as an expression promoter for specific expression of the introduced gene in the β-cell of the animal. The portion from −715 bp to +31 bp was obtained by PCR using the primers described below using a rat genome DNA as a template:

5′-primer:

(SEQ ID No. 3)

5′-TCTCAACTCCTTGAAAATAGCTACCT-f-3′
3′-primer:

(SEQ ID No. 4)

-GGTCTATGATTGTAGCTGGTCACTTA-r-3′

Platinum Pfx DNA polymerase (manufactured by Invitrogen Co.) was used as a DNA polymerase.

(Expression cDNA)

Human SREBP-2 cDNA expression vector pCMVh SREBP-2 (an expression vector of cDNA containing a transfer active portion (1 to 460 amino acid residues) of human SREBP-2; Amemiya-Kudo M, Shimano H, Hasty A H, Yahagi N, Yoshikawa T, Matsuzaka T, Okazaki H, Tamura Y, Iizuka Y, Ohashi K, Osuga J, Harada K, Gotoda T, Sato R, Kimura S, Ishibashi S, and Yamada N, J. Lipid Res. 2002, 43(8), 1220-35) was used as the cDNA of human SREBP (sterol regulatory element-binding protein).

Poly A signal of human growth hormone in the expression vector was used for the poly A signal for terminating transcription. The DNA was fused in plasmid blue script II SK(+/−) (manufactured by Stragene) as shown in FIG. 1 to construct an expression vector. The base sequence of the DNA portion obtained by PCR was confirmed by DNA sequencing. The function of the expressed vector was confirmed by Luciferase reporter assay of the transcription activity of SREBP-2 transfected into a cell line of the β-cell and by western blotting of the expressed protein.

### (2) Preparation of Microinjection DNA

The plasmid DNA was purified in large scale using a plasmid purification kit (manufactured by Quiagen Co.), and the purified DNA was cleaved with restriction enzymes NotI and XhoI. The injected DNA portion shown in FIG. 1 was separated by agarose electrophoresis, and was purified using QIAEX II Gel Extraction Kit (manufactured by Quiagen Co.).

### (3) Preparation of Transgenic Mouse

The transgenic mouse was prepared according to a published report (Method in Enzymology, Vol. 225, “Guide Techniques in Mouse Development”, edited by Paul M. Wassarman, Melvin L. DePamphilis, Academic Press, Inc.). An outline of the preparation method comprises: preparing the expression gene obtained in (1) in male pronuclei of the fertilized ovum (BDF2) obtained by natural mating between B6SJL/F2 mice; introducing about 1000 copies of the expression gene by a micro-injection method (based on U.S. Pat. No. 4,873,191 by Ohio University); and transplanting the expression vector into the uterine tube in an ICR pseudo-pregnant mouse (0.5 dpc). The survived infant mouse was extracted by Caesarean section or by spontaneous delivery from the pregnant mouse at 19.5 dpc after transplantation to obtain 45 F0 mice.

Introduction of injected gene into the genome DNA of five F0 mice was confirmed by PCR as described hereinafter by extracting the tail DNA from 45 mice obtained. The PCR method was also used for screening of genotypes.

Particularly, the following primer sets specific to the introduced gene portion were used for the reaction under the following conditions using the tail DNA as a template:

### Primer:

(hBP-2 side, sense):

(SEQ ID No. 5)

5′AGCTTCTAAAGGGCATCGACCTA3′
(hGH side, antisense):

(SEQ ID No. 6)

5′TAGAGGACACCTAGTCAGACAAAATGAT3′

### PCR Condition:

94° C.×4 min.×1 cycle

94° C.×1 min.×35 cycle

60° C.×1 min.×35 cycle

72° C.×1 min.×35 cycle 72° C.×4 min.×1 cycle

### (4) Identification of Expression Line

As the result, C57 BL6/J mice (purchased from CLEA Co.) were mated with five founder mice in which introduction of the gene into the chromosome DNA was confirmed. The mouse line was established by confirming introduction of the gene into descendants by PCR of the tail DNA as described above.

Langerhans' islet was separated from the pancreas of each descendant mouse line, total RNA was extracted using TRIzol (trade name, manufactured by Invitrogen Co.), and expression of human SREBP-2 was investigated by a real-time PCR method. Line A exhibiting most frequent expression was established, the mouse of the established line was repeatedly mated with C57BL6/J mouse, and backcrossing was repeated so that breeding and genetic background are close to those of C57BL6/J strain.

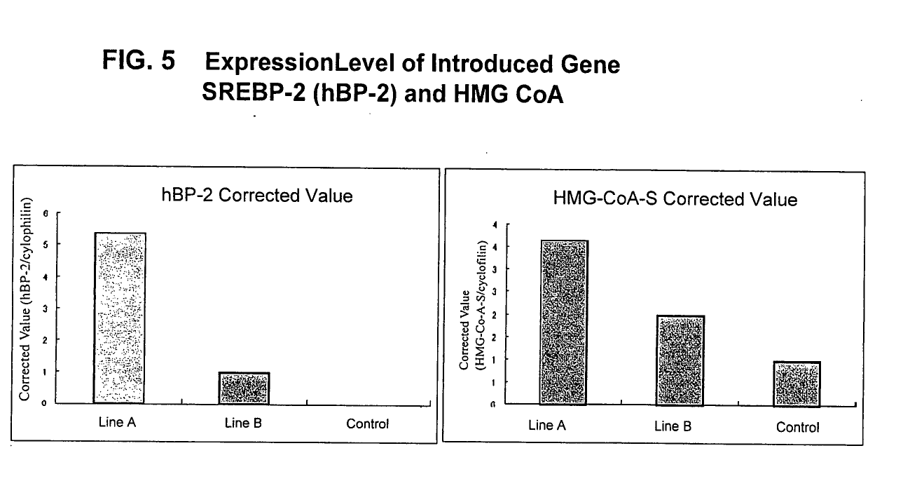

During the period of mating, the transgenic mouse obtained by genotyping and non-transgenic littermate mouse were subjected to initial analysis. Transgenic mouse line A was stable and showed a positive ratio of about 50% in the littermate according to Mendel's law. It was confirmed that integration of the introduced gene is stably propagated in the chromosome. Expression of human SREBP-2 mRNA was stably confirmed without dispersion among the individuals by measuring the expression using a qualitative real-time PCR (the method is described later, see FIG. 5) from the RNA derived from Langerhans' islet (isolation method is described later), and the transgenic mouse line excessively expressing β-cell specific human SREBP-2 was considered to be established.

Qualitative real-time PCR was performed as follows. RNA of each sample was extracted using TRIzol, and cDNA was synthesized from the RNA using Thermoscript (manufactured by Invitrogen Co.) by quantitative PCR by TaqMan (Applied Biosystems, ABI) (40 cycles of 50° C./2 minutes and 95° C./10 minutes followed by 95° C./15 seconds and 60° C./1 minute) using ABI Prism 7000 PCR instrument (trade name, manufactured by Applied Biosystems Co.). The primers and probes used were as follows.

| | |
|---|---|
| Human SREBP-2: | |
| 5′-CCAACTCTGCAAGTCAAGGTTTCT-f-3′ | (SEQ ID No. 7) |
| 5′-GCGTGATCATTACCGTCTGTTGT-r-3′ | (SEQ ID No. 8) |
| Mouse HMG CoA synthetase: | |
| 5′-AGGAAACTTCGCTCACACCT-f-3′ | (SEQ ID No. 9) |
| 5′-GCCATGTATCTGTTTTGGCC-r-3′ | (SEQ ID No. 10) |
| Probe: | |
| CAGCAGCCCAGCAGAGGTTTTCTACAATC | (SEQ ID No. 11) |
| Cyclophilin: | |
| 5′-TGGCTCACAGTTCTTCATAACCA-f-3′ | (SEQ ID No. 12) |
| 5′-ATGACATCCTTCAGTGGCTTGTC-r-3′ | (SEQ ID No. 13) |
| Probe: | |
| 5′-TCCATGCCCTCTAGAACTTTGCCGAA-3′ | (SEQ ID No. 14) |

### (5) Phenotype of Prepared Mouse: Confirmation of Diabetes

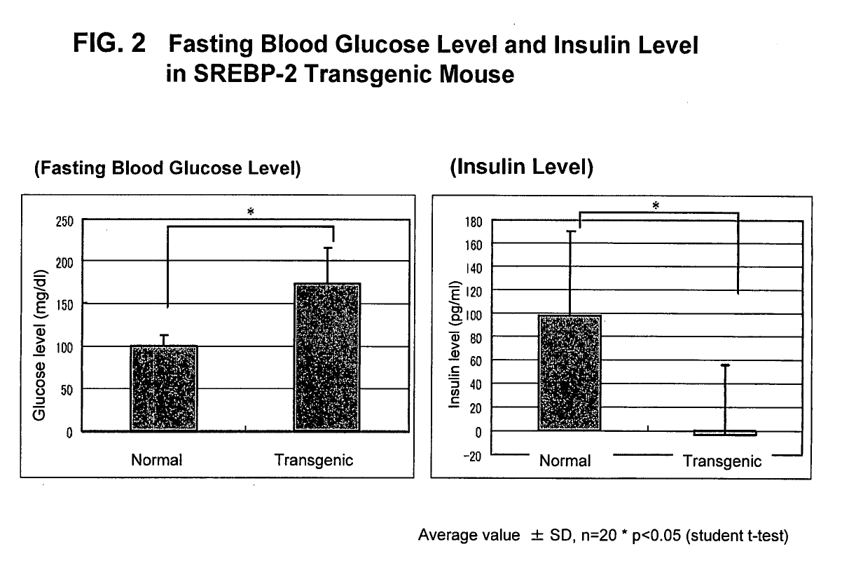

Positive urine sugar and polyuria were observed in the transgenic mouse line A obtained as described above during breeding with normal diet, and onset of diabetes was suggested. As shown in FIG. 2, the measurement of fasting glucose level (glucose measuring kit, manufactured by Wako Pure Chemical Industries, Ltd.) showed a significantly higher level of 174 mg/dl in the transgenic mouse group as compared with the level of 99 mg/dl in the non-transgenic littermate reference group, and the insulin level was below the measuring sensitivity (ELISA kit, manufactured by Shibayagi Co.). The blood glucose level under daily feeding evidently showed a level of 300 to 500 mg/dl corresponding to diabetes.

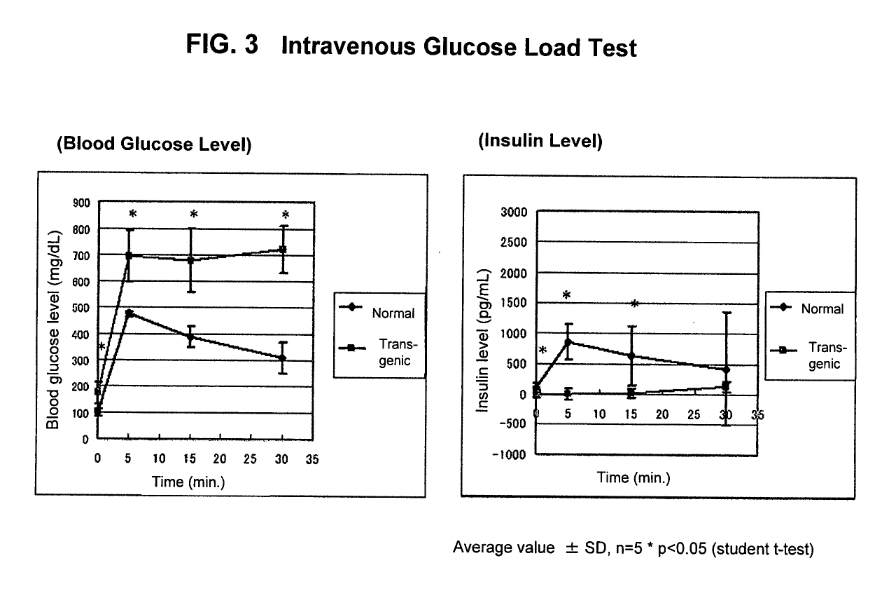

In the glucose load test of the transgenic mouse group (glucose was injected into the vein of the tail for glucose load test (1 g/kg, 20% aqueous solution), the blood was sampled from the vein of the orbit with time (before injection and 5, 15 and 30 minutes after injection), and blood glucose level and blood insulin level were measured. The time-dependent pattern showed a diabetes pattern of the impaired insulin secretion type (FIG. 3). The level was significantly higher in the transgenic mouse than in the control at each time point, and the high glucose level after the load of glucose persisted. Increase of the blood insulin level corresponding to elevation of the blood glucose level, which was observed in the reference group, was scarcely observed, and the transgenic mouse group showed evidently impaired secretion of insulin.

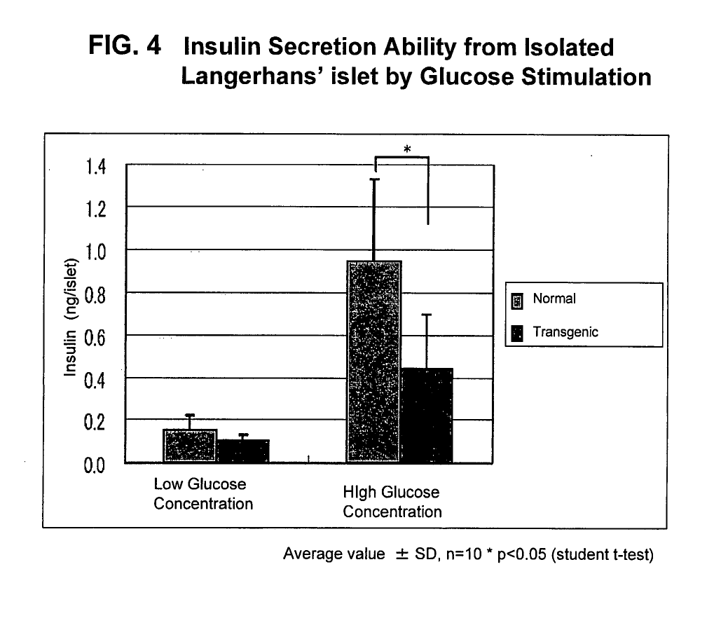

When Langerhans' islet was separated for directly confirming impaired secretion of insulin, the size of each islet was reduced to about 50% with abnormal shape of the islet, which showed impairment of Langerhans' islet. When insulin secretion ability of the Langerhans' islet cells during cultivation was measured by making the size of the cells uniform after separation, the amount of insulin secretion against the concentration of glucose in the culture medium showed 50% decrease as shown in FIG. 4, and decline of the insulin secretion ability was observed for respective Langerhans' islets.

Langerhans' islet was isolated as follows. The mouse was slaughtered by dislocation of the cervical spine, and 2.5 ml of a collagenase solution (4 mg/ml, 10 mM HEPES (pH 7.4), 0.5% BSA (KRBH)) was injected into the pancreatic duct after ligating the bile duct. The swelled pancreas was further cultivated at 37° C. for 3.5 minutes and washed with KRBH, and isolated Langerhans' islet was collected under a stereoscopic microscope.

It was confirmed in the established human SREBP-2 transgenic mouse using the rat insulin promoter that the mouse developed diabetes due to impaired secretion of insulin. For investigating the amount of expression of each line, as shown in FIG. 5, a part of the same investigation was performed with respect to line B in which the amount of expression of human SREBP-2 is smaller than line A, and it was confirmed that each investigation item concerning diabetes slightly exists in response to the smaller amount of expression of human SREBP-2. In other words, it was confirmed that the mouse exhibits a phenotype depending on the amount of expression of the introduced SREBP-2 gene, and the phenotype of line A was caused by the effect depending on the amount of the expressed SREBP-2 gene, not by the non-specific effect by introducing the gene.

### (6) Disease Condition of Diabetes of Prepared Mouse

The transgenic mouse prepared as described above manifests the disease condition of type 2 diabetes in that secretion response to simulation with glucose rather than basal secretion is impaired, not by depletion of insulin by abolition of the β-cell as seen in type 1 diabetes. The body weight, appearance and the weight of the anatomical adipose tissue of the mouse are almost identical to those of the normal mouse, and no obesity is observed. In other words, the disease condition of this mouse model is evidently different from that of the obese mouse model and insulin resistant mouse model, although these models equally suffers from type 2 diabetes, and the model mouse of this invention serves as the type 2 diabetes model mouse of the non-obese and insulin secretion impaired type that is frequently found in Japanese. As shown in FIG. 5, expression of HMG CoA synthetase as a key enzyme in the cholesterol synthesis system is induced depending on the amount of expression of the introduced gene. Accordingly, a relation between the expression of HMG CoA synthetase and onset of diabetes was conjectured since cholesterol synthesis in the β-cell is supposed to be activated in the mouse of the invention.

The transgenic mouse prepared as described above suffers from diabetes due to a novel mechanism of impaired secretion of insulin, and encompasses new concept of onset of diabetes. Accumulation of neutral lipids in β-cell has been noticed as pathogenesis of impaired secretion of insulin. The mouse of the invention that is anticipated to accumulate cholesterol may be a quite novel diabetes model that suggests a possibility of quite new pathological mechanism of impairment of metabolism of cholesterol and secretion of insulin.

### INDUSTRIAL APPLICABILITY

The non-human transgenic animal of the invention is useful as a tool for investigating the onset mechanism of type 2 diabetes and as a tool for screening therapeutic agents of type 2 diabetes. The transgenic mouse according to a preferable embodiment of the invention is advantageous for investigations since a half of descendants can be used as diseased mice and remaining half of the descendants can be used as normal reference group after breeding. Other known model animals that exhibit disease conditions resembling to type 2 diabetes have been inconvenient for use since the animals evidently manifests obesity or special loading diets such as high fat diets should be given for a long period of time in order to allow the animals to develop diabetes since insulin resistance is emphasized. The mouse of the invention is a quite convenient model in that the mouse is not obese and develops diabetes by lowered level of insulin secretion since younger generations even by being fed on normal diet. It is also advantageous that severe diabetes is developed by feeding on loading diet.

The screening method according to another embodiment of the invention can be effectively used for screening therapeutic agents of type 2 diabetes abundant in Japanese.

1. A non-human transgenic animal as a model of type 2 diabetes manifesting a symptom of type 2 diabetes by excessive expression of the active SREBP-2 protein in pancreatic β-cells by introducing a recombinant DNA in which a DNA encoding the active SREBP-2 protein is disposed under the control of a promoter.

2. The transgenic animal according to claim 1, wherein the symptom of type 2 diabetes includes abnormal cholesterol metabolism and impaired insulin secretion in the pancreatic β-cells.

3. The transgenic animal according to claim 1, wherein the DNA encoding the active SREBP-2 protein is a human active SREBP-2 cDNA.

4. The transgenic animal according to claim 1, wherein the promoter is a promoter of rat insulin I gene.

5. The transgenic animal according to claim 1, wherein a gene marker is further introduced into the recombinant DNA.

6. The transgenic animal according to claim 5, wherein the gene marker is a green fluorescent protein.

7. The transgenic animal according to claim 1, wherein the animal is mouse, rat or rabbit.

8. The transgenic animal according to claim 7, wherein the animal is mouse.

9. A method for screening a therapeutic agent for type 2 diabetes using the transgenic animal according to claim 1.

10. The method of screening a therapeutic agent for type 2 diabetes according to claim 9, comprising the step of observing the change of the symptom of diabetes after administering a test compound to the transgenic animal.

11. A method for preparing a transgenic animal manifesting the symptom of type 2 diabetes comprising the steps of:
(i) constructing a recombinant DNA in which a DNA encoding a human active SREBP-2 protein is disposed under the control of a promoter;
(ii) introducing the recombinant DNA and a gene marker into fertilized ovum of a non-human animal;
(iii) transplanting the fertilized ovum into a pseudo-pregnant non-human mammal to breed the mammal; and
(iv) selecting infants having the recombinant DNA from one or more delivered infants using an expression product of the gene marker as an index.

12. The method according to claim 11, wherein the symptom of type 2 diabetes includes abnormal cholesterol metabolism and impaired insulin secretion in the pancreatic β-cells.

13. The method according to claim 11, wherein the promoter is a promoter of rat insulin I gene.

14. The method according to claim 11, wherein a gene marker is further introduced into the recombinant DNA.

15. The method according to claim 11, wherein the gene marker is a green fluorescent protein.

16. The method according to claim 11, wherein the animal is mouse, rat or rabbit.

17. The method according to claim 16, wherein the animal is mouse.
